(19) United States
(12) Patent Application Publication
(10) Pub. No.: US 2004/0092762 A1
(43) Pub. Date: May 13, 2004

(54) Polyhaloalkylaryls

(76) Inventors: Albrecht Marhold, Leverkusen (DE); Axel Pleschke, Koln (DE)

Correspondence Address: BAYER CHEMICALS CORPORATION, PATENT DEPARTMENT, 100 BAYER ROAD, PITTSBURGH, PA, 15205-9741, US

(21) Appl. No.: 10/703,835

(22) Filed: Nov. 7, 2003

(30) Foreign Application Priority Data
Nov. 11, 2002 (DE) 10252273.1

(51) Int. Cl. C07C 235/34; C07C 237/38

(52) U.S. Cl. 560/19; 564/163; 564/442

(57) ABSTRACT

The present invention relates to polyhaloalkylaryls, to a process for preparing them and to the use of the polyhaloalkylaryls for preparing active ingredients.

### BACKGROUND OF THE INVENTION

1. Field of the Invention

The present invention relates to polyhaloalkylaryls, to a process for preparing them and to the use of the polyhaloalkylaryls for preparing active ingredients, in particular in agrochemicals and pharmaceuticals.

2. Brief Description of the Prior Art

Polyhaloalkylaryls are valuable starting products for the preparation of active ingredients in agrochemicals and pharmaceuticals, since the polyhaloalkyl substituents increase the lipophilicity and therefore the membrane permeability of the entire active ingredient molecule. For example, substituted 4-perfluoroalkylanilines are particularly suitable for preparing effective insecticides of the aroylurea type (see also EP-A 919 542 and EP-A 936 212).

The processes for preparing polyhaloalkylaryls and their disadvantages are described as follows. Perfluoroalkylaryls can be prepared, for example, by reacting aromatics with perfluoroalkyl iodides or bromides in aprotic solvents, either in the presence of metals and sulphur dioxide (EP-A 206 951 and FR-A 2 660 923) or in the presence of alkali metal dithionite (EP-A 298 803). Similarly, perfluoroalkyl chlorides can be reacted in the presence of dimethyl sulphoxide (Huang et al., J. Fluorine Chem., 111, 2001, 107-113).

A disadvantage of the methods mentioned above is that the reaction has to be in an aprotic polar solvent, in particular dimethylformamide or dimethyl sulphoxide, which, as a consequence of their high boiling point, are difficult to remove from the products and are barely recyclable, and are additionally physiologically unacceptable. Moreover, all methods have only moderate yields.

In a process according to EP-A 1 006 102, perfluoroalkylanilines can be obtained by reacting anilines with perfluoroalkyl iodides in a biphasic system in the presence of a reducing agent. However, the perfluoroalkyl iodides are not only expensive, but also, as a consequence of their high molecular weight, cause low atom economy.

There is therefore a need for a process which enables the preparation of polyhaloalkylaryls in good yields and in a simple manner.

### SUMMARY OF THE INVENTION

In accordance with the foregoing, the present invention encompasses a process for preparing compounds of the formula (I)

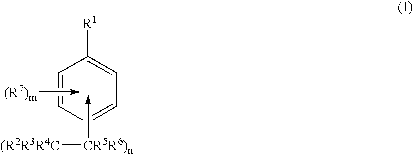

in which

R¹ is C₁-C₁₂-alkyl, NR⁸R⁹ or OR¹⁰, where R⁸, R⁹ and R¹⁰ are each independently hydrogen, C₁-C₁₂-alkyl, CO(C₁-C₁₂-alkyl), CO(C₅-C₁₄-aryl), CO(C₆-C₁₅-arylalkyl), COO(C₁-C₁₂-alkyl), COO(C₅-C₁₄-aryl), COO(C₆-C₁₅-arylalkyl), COO(C₂-C₁₂alkenyl), CONH(C₁-C₁₂-alkyl), CONH(C₅-C₁₄-aryl), CONH(C₆C₁₅-arylalkyl), CON(C₁-C₁₂-alkyl)₂, CON(C₅-C₁₄-aryl)₂, CON(C₆-C₁₅-arylalkyl)₂ or C₆-C₁₅-arylalkyl, or NR⁸R⁹ as a whole is a cyclic radical having a total of 4 to 16 carbon atoms and

R², R³, R⁴, R⁵ and R⁶ are each independently hydrogen, fluorine, chlorine, bromine or C₁-C₁₂-polyfluoroalkyl, and/or at least two of the R², R³, R⁴, R⁵ and R⁶ radicals each form one or more cyclic polyfluoroalkyl radicals each having a total of 4 to 20 carbon atoms, with the proviso that in all cases the sum of the fluorine atoms on the carbon atom which forms the bond to the aromatic ring and the fluorine atoms on the adjacent carbon atom or atoms is at least two and

n is one or two, and

R⁷ is C₁-C₁₂-alkyl, C₅-C₁₄-aryl, C₆-C₁₅-arylalkyl, hydroxyl, chlorine, bromine, fluorine, nitro, cyano, free or protected formyl, C₁-C₁₂-haloalkyl, or radicals of the formulae (IIa) to (IIf),

A—B—D—E (IIa)

A—E (IIb)

A—SO₂—E (IIc)

A—B—SO₂R¹¹ (IId)

A—SO₃W (IIe)

A—COW (IIf)

in which, each independently,

A is absent or is a C₁-C₈-alkylene radical and

B is absent or is oxygen, sulphur or NR¹²,

where

R¹² is hydrogen, C₁-C₈-alkyl, C₆-C₁₅-arylalkyl or C₅-C₁₄-aryl, and

D is a carbonyl group and

E is R¹³, OR¹³, NHR¹¹ or N(R¹¹)₂

where

R¹³ is C₁-C₈-alkyl, C₆-C₁₅-arylalkyl, C₁-C₈-haloalkyl or C₅-C₁₄-aryl, and

R¹¹ is in each case independently C₁-C₈-alkyl, C₆-C₁₅arylalkyl or C₆-C₁₄-aryl, or N(R¹¹)₂ together is a cyclic amino radical having 4 to 12 carbon atoms and

W is OH, NH₂ or OM where M is an alkali metal ion, half an equivalent of an alkali earth metal ion, an ammonium ion or an organic ammonium ion, or

in each case two R⁷ radicals together may form a cyclic radical having a total of 5 to 12 carbon atoms, and

m is an integer from 0 to 5-n,

which is characterized in that compounds of the formula (II)

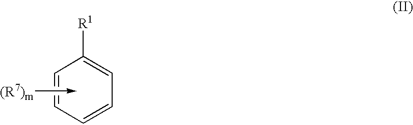

in which

R¹, R⁷ and m are each as defined above are reacted with compounds of the formula (III)

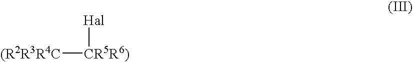

in which

R², R³, R⁴, R⁵ and R⁶ are each as defined above and

Hal is bromine or chlorine, preferably bromine, and the reaction is effected

in a multiphasic reaction medium which has one aqueous phase and at least one, preferably exactly one, organic phase and

in the presence of phase transfer catalyst and

in the presence of a reducing agent and/or light having a wavelength of 400 nm or less and optionally in the presence of a base.

For the purposes of the invention, all radical definitions, parameters and illustrations hereinabove and listed hereinbelow, in general or within areas of preference, i.e. the particular ranges and preferred ranges, may be combined as desired.

### DETAILED DESCRIPTION OF THE INVENTION

Alkyl, alkylene, alkoxy and alkenyl are each independently a straight-chain, cyclic, branched or unbranched alkyl, alkylene, alkoxy or alkenyl radical respectively. The same applies to a nonaromatic moiety of an arylalkyl radical.

C₁-C₄-Alkyl is, for example, methyl, ethyl, n-propyl, isopropyl, n-butyl, sec-butyl and tert-butyl, C₁-C₈-alkyl is additionally, for example, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, 1-ethyl-2-methylpropyl, n-heptyl and n-octyl, and C₁-C₁₂-alkyl is further additionally, for example, adamantyl, n-nonyl, n-decyl and n-dodecyl.

C₁-C₈-Alkoxy is, for example, methoxy, ethoxy, n-propoxy, isopropoxy, n-butoxy, sec-butoxy and tert-butoxy, n-pentoxy, 1-methylbutoxy, 2-methylbutoxy, 3-methylbutoxy, neopentoxy, 1-ethylpropoxy, cylcohexoxy, cyclopentoxy, n-hexoxy and n-octoxy, and C₁-C₁₂-alkoxy is additionally, for example, adamantoxy, the isomeric methoxy radicals, n-decoxy and n-dodecoxy.

C₂-C₂₀-Alkenyl is, for example, vinyl, 1-propenyl, isopropenyl, 1-butenyl, 2-butenyl, 1-pentenyl, 2-pentenyl, 2-methyl-1-butenyl, 2-methyl-2-butenyl, 3-methyl-1-butenyl, 1-hexenyl, 1-heptenyl, 1-octenyl or 2-octenyl.

Polyfluoroalkyl is in each case independently a straight-chain, cyclic, branched or unbranched alkyl radical which is substituted by at least two fluorine atoms and optionally further by chlorine atoms and/or bromine atoms.

For example, C₁-C₁₂-polyfluoroalkyl is trifluoromethyl, difluorochloromethyl, penafluoroethyl, 1,1-dichloro-2,2,2-trifluoroethyl, heptafluoroisopropyl, n-nonafluorobutyl, perfluorocyclopentyl, perfluorocyclohexyl and perfluorododecyl.

Perfluoroalkyl is in each case independently a straight-chain, cyclic, branched or unbranched alkyl radical which is fully substituted by fluorine atoms.

Aryl is in each case independently a heteroaromatic radical having 5 to 14 framework carbon atoms of which no, one, two or three framework carbon atoms per cycle, but at least one framework carbon atom in the entire molecule may be substituted by heteroatoms selected from the group of nitrogen, sulphur and oxygen, but is preferably a carbocyclic aromatic radical having 6 to 14 framework carbon atoms.

Examples of carbocyclic aromatic radicals having 6 to 14 framework carbon atoms are phenyl, naphthyl, phenanthrenyl, anthracenyl or fluorenyl; heteroaromatic radicals having 5 to 14 framework carbon atoms of which no, one, two or three framework carbon atoms per cycle, but at least one framework carbon atom in the entire molecule, may be substituted by heteroatoms selected from the group of nitrogen, sulphur or oxygen are, for example, pyridinyl, oxazolyl, benzofuranyl, dibenzofuranyl or quinolinyl.

The carbocyclic aromatic radical or heteroaromatic radical may also be substituted by up to five identical or different substituents per cycle which are selected from the group of chlorine, fluorine, C₁-C,₂-alkyl, C₁-C₁₂-perfluoroalkyl, COO(C₁-C₈-alkyl), CON(C₁-C₈-alkyl)₂, COO(C₁-C₈-arylalkyl), COO(C₄-C₁₄-aryl), CO(C₁-C₈-alkyl), C₅-C₁₅-arylalkyl or tri(C₁-C₆-alkyl)siloxyl.

Arylalkyl is in each case independently a straight-chain, cyclic, branched or unbranched alkyl radical which may be singly, multiply or fully substituted by aryl radicals as defined above.

C₆-C₁₅-Arylalkyl is, for example and with preference, benzyl.

The preferred substitution patterns for compounds of the formulae (I), (II) and (III) are defined hereinbelow:

R¹ is preferably NR⁷R⁸, and NR⁷R⁸ is NH₂ or NHCO(C₁-C₁₂-alkyl) and preferably NH₂.

R², R³, R⁴, R⁵ and R⁶ are preferably each hydrogen, chlorine, fluorine or C₁-C₄-perfluoroalkyl, or R²R³R⁴C—CR⁵R⁶ which is a cyclic polyfluoro alkyl radical having a total of 4 to 12 carbon atoms.

More preferably, R²R³ R⁴C—CR⁵R⁶ is heptafluoro-2-propyl, 1-bromo-1,1,2,3,3,3-hexafluoro-2-bromo-1,1,2,2-tetrafluoroethyl, 2-chloro-1,1,2,2-tetrafluoroethyl, 1-chloro-1,1,2,3,3,3-hexafluoro-2-propyl, 2-bromo-2-chlorotrifluoroethyl, 2-bromo-1-chlorotrifluoroethyl, 3-bromo-2,3-dichloro-1,1,1,4,4,4-hexafluoro-2-butyl, 2-chloro-3,3,4,4-tetrafluorocyclobutyl, 2-bromo-3,3,4,4-tetrafluorocyclobutyl 2-chloro-3,3,4,4,5,5-hexafluorocyclopentyl and 2-bromo-3,3,4,4,5,5-hexafluorocyclopentyl.

n is preferably 1.

R⁷ is preferably in each case independently C₁-C₄-alkyl, chlorine, fluorine, nitro, cyano or C₁-C₄-alkoxy, more preferably methyl, ethyl, methoxy or ethoxy, most preferably methyl.

m is preferably 1 or 2, and more preferably 1.

Particularly preferred compounds of the formula (I) are 2-methyl-4-(heptafluoro-2-propyl)aniline, N,2dimethyl-4-(1,1,1,2,3,3,3-heptafluoro-2-propyl)aniline, 2-methyl-4-(1-bromo-1,1,2,3,3,3-hexafluoro-2-propyl)aniline, 2-methyl-4-(2-bromo-1,1,2,2-tetrafluoroethyl)aniline, 2-methyl-4-(2-chloro-1,1,2,2-tetrafluoroethyl)aniline, 2-methyl-4-(1-chloro-1,1,2,3,3,3-hexafluoro-2-propyl)aniline, 2-methyl-4-(2-bromo-2-chlorotrifluoroethyl)aniline, 2-methyl-4-(2-bromo-1-chlorotrifluoroethyl)aniline, 2-methyl-4-(3-bromo-2,3-dichloro-1,1,1,4,4,4-hexafluoro-2-butyl)aniline, 2-methyl-4-(2-chloro-3,3,4,4-tetrafluorocyclobutyl)aniline, 2-methyl-4-(2-chloro-3,3,4,4,5,5-hexafluorocyclopentyl)aniline, 2-methyl-4-(2-bromo-3,3,4,4-tetrafluorocyclobutyl)aniline, 2-methyl-4-(2-bromo3,3,4,4,5,5-hexafluorocyclopentyl)aniline, 2-methyl-4-(1-bromo-1,1,2,3,3,3-hexafluoro-2-propyl)acetanilide, 2-methyl-4-(2-bromo-1,1,1,2,3,4,4,4-octafluoro-3-butyl)aniline and 2-methyl-4-(2-bromo-2,3,3,4,4,5,5-octafluorocyclo-1-pentyl)aniline.

Preferred compounds of the formula (III) are heptafluoro-2-bromopropane, heptafluoro-2-chloropropane, 1,2-dibromotetrafluoroethane, 1,2-dibromo-1-chlorotrifluoroethane, 2,3-dibromooctafluorobutane, 2,3-dibromo-2,3-dichlorohexafluorobutane, 2,3-dibromo-2,3dichlorohexafluorobutane, 2,3-dibromo-1,1,1,3,4,4,4-heptafluorobutane, 2,3-dibromo-2-chloro-1,1,1,4,4,4-hexafluorobutane, 1,2-dibromohexafluoropropane and 1,2-dichlorohexafluoropropane, and particular preference is given to heptafluoro-2-bromopropane, 1,2-dibromohexafluoropropane, 2-dibromo-1-chlorotrifluoroethane. Very particular preference is given to 1,2-dibromohexafluoropropane and heptafluoro-2-bromopropane.

The molar ratio of compounds of the formula (III) to compounds of the formula (II) per equivalent of n may be, for example, 0.7 to 1.8, preferably 0.9 to 1.2 and more preferably 1.0 to 1.1

The compounds of the formula (III) which are used as starting products are known from the literature or can be synthesized analogously to the literature.

The process according to the invention is carried out in a multiphasic reaction medium which has one aqueous and at least one organic phase.

Particularly suitable organic solvents for multiphasic reaction media are, for example, aliphatic or aromatic, optionally halogenated hydrocarbons, for example benzine fractions, benzene, toluene, xylene, chlorobenzene, dichlorobenzene, petroleum ether, hexane, cyclohexane, dichloromethane, chloroform or carbon tetrachloride, ethers, for example diethyl ether, diisopropyl ether, tert-butyl methyl ether, ketones, for example cyclohexanone, butanone or methyl isobutyl ketone, and esters, for example methyl acetate or ethyl acetate.

Moreover, the process according to the invention is carried out in the presence of phase transfer catalysts.

Suitable phase transfer catalysts are, for example, crown ethers such as 18-crown-6, 12-crown4, dibenzo-18-crown-6 or dibenzo-12-crown-4, cryptands such as cryptand[2.2.2] or podands such as polyglycol ethers or those of the formula (IV),

(cation⁺)(anion⁻) (IV)

in which

(cation⁺) is a substituted quaternary ammonium or phosphonium cation and

(anion⁻) is the anion of an organic or inorganic acid.

Preferred phase transfer catalysts are those of the formula (IV) in which (cation⁺) is a cation of the formula (V)

[pnic(C₁-C₁₂-alkyl)_{q}(C₆-C₁₅-arylalkyl)ᵣ(C₅-C₁₄-aryl)ₛ({(C₂-C₆-alkyl)-O]ᵥ—(C₁-C₆-alkyl)}ₜ)]⁺ (V)

in which

pnic is nitrogen or phosphorus and

in which in each case (q+r+s+t)=4.

(Anion⁻) in formula (IV) is preferably fluoride, chloride, bromide, iodide, acetate, nitrate, sulphate, hydrogensulphate, tetrafluoroborate, hexafluorophosphate, tosylate and triflate, more preferably chloride, bromide, iodide, sulphate and hydrogensulphate.

Particularly preferred phase transfer catalysts are tetra-n-butylammonium iodide, tetra-n-butylammonium bromide, tetra-n-butylammonium hydrogensulphate, tetra-n-butylammonium chloride, tributylmethylphosphonium bromide, trimethyl-C₃/C₁₅-alkylammonium chloride, trimethyl-C₁₃/C₁₅-alkylammonium bromide, dibenzyldimethylammonium methylsulphate, dimethyl-C₁₂/C₁₄-alkylbenzylammonium chloride, dimethyl-C₁₂/C₁₄-alkylbenzylammonium bromide, triethylbenzylammonium chloride, methyltrioctylammonium chloride, trimethylbenzylammonium chloride, tetrakisdiethylaminophosphonium chloride, bromide or iodide, and also tris-[2-(2-methoxyethoxy)ethyl]amine, and very particular preference is given to tetra-n-butylammonium hydrogensulphate.

The reaction temperature may be, for example, −10° C. up to the boiling point of the reaction medium under reaction pressure, up to a maximum of 200° C. Preferably the reaction temperature is from 0 to 70° C.

The reaction pressure may be, for example, 0.5 to 100 bar, and preferably ambient pressure.

The process according to the invention is further carried out in the presence of a reducing agent and/or in the presence of light having a wavelength of 400 nm or less.

Suitable reducing agents are, for example, sulphur compounds in the average formal oxidation states +III, +IV and +V, optionally in a mixture with a metal which has a standard reduction potential of 0 V or less.

Such sulphur compounds are, for example, alkali metal dithionites, such as sodium dithionite, potassium dithionite, or sulphur dioxide.

Suitable metals are, for example, manganese, zinc or aluminium.

Particularly suitable light sources which generate light having a wavelength of 400 nm or less are all customary UV lamps, in particular mercury vapour lamps.

Particular preference is given to carrying out the process according to the invention in the presence of alkali metal dithionite, with very particular preference in the presence of sodium dithionite.

Optionally, but preferably, the process according to the invention is carried out in the presence of base.

Suitable bases are, for example: alkali earth metal or alkali metal hydroxides, acetates, phosphates, hydrogen phosphates, carbonates or hydrogen carbonates, for example sodium hydroxide, potassium hydroxide, sodium acetate, potassium acetate, calcium acetate, sodium carbonate, potassium carbonate, potassium hydrogencarbonate or sodium hydrogencarbonate, ammonium salts, for example ammonium acetate, ammonium carbonate, amines, for example trimethylamine, triethylamine, tributylamine, diisopropylethylamine, tetramethylguanidine, N,N-dimethylaniline, diazabicyclooctane (DABCO), diazabicyclononene (DBN), diazabicycloundecene (DBU), N-methylpiperidine and piperidine, or aromatic nitrogen compounds, for example pyridine, 2-, 3- and 4-N,N-dimethylaminopyridine, and preference is given to alkali metal hydroxides, carbonates and hydrogencarbonates.

Ionic fluorides are, for example, quaternary ammonium fluorides or phosphonium fluorides, and also alkali metal fluorides or mixtures of the compounds mentioned.

Examples of ammonium fluorides or phosphonium fluorides are those of the formula (VI)

(cation⁺)(F⁻) (VI)

in which the (cation+) is as defined under the formula (IV), including its areas of preference.

Optionally, mixtures of phase transfer catalysts as defined above and/or halex catalysts with alkali metal fluorides can also be used.

Preferred alkali metal fluorides are sodium fluoride, potassium fluoride and caesium fluoride or mixtures thereof, and particular preference is given to potassium fluoride.

Halex catalysts are, for example, tetrakis(dialkylamino)phosphonium compounds (WO 98/05610) or compounds of the formula VII)

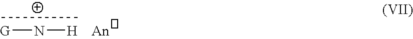

in which

G is a radical of the formulae (VIIIa) or (VIIIb) 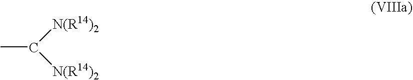 —P{N(R¹⁴)₂}₃ (VIIIb)

and

H, independently of G, is a radical of the formulae (VIIIa), (VIIIb), (VIIIc) or (VIIId) 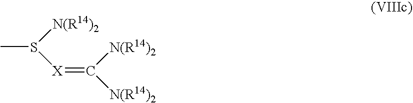 —S[N(R¹⁴)₂]₂ (VIIId)

where

the R¹⁴ radicals are each independently C₁-C₁₂-alkyl, C₂-C₁₀-alkenyl or C₆-C₁₂-aryl, or

N(R¹¹)₂ e is a 3- to 5-membered, saturated or unsaturated ring, or

the radicals of the formula (VIIIa) and/or the group

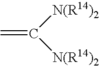

as a whole may each be a saturated or unsaturated, 4- to 8-membered ring, and

X is nitrogen or phosphorus and

An^{⊖} is one equivalent of an anion, for example and with preference chloride, bromide, (CH₃)₃SiF₂^{⊖}, HF₂^{⊖}, H₂F₂^{⊖}, tetrafluoroborate, hexafluorophosphate, carbonate or sulphate.

The compounds of the formula (VII) are obtainable, for example, by reacting compounds of the formula (IX)

[G—An′]^{⊕ n} [symbol]^{⊖} (IX)

in which

G and An^{⊖} are each as defined in formula (X) and

An′ is chlorine or bromine

with compounds of the formula (X)

HN═G′ (X)

in which

G′, with regard to the arrangement of the atoms, is as defined for G in formula (X), but is divalent, and the reaction is effected in the presence of a base.

The halex catalysts of the formula (VII) are described in DE 10129 057.

The molar ratio of ionic fluoride to bromine or chlorine atoms in compounds of the formula (I) used may be, for example, 0.7 to 5, preferably 0.9 to 2 and more preferably 1.1 to 1.7. The amount of ionic fluoride in principle has no upper limit, but larger amounts are uneconomic.

It has been found that bromine atoms are typically more rapidly exchanged than chlorine atoms and the substitution rate increases in the order tertiary, secondary, primary carbon atoms.

Preference is given to carrying out the halogen exchange in the presence of organic solvent. Suitable organic solvents are, for example: ketones such as acetone, 2-butanone or methyl isobutyl ketone; nitriles, for example acetonitrile, propionitrile, benzonitrile, benzyl nitrile or butyronitrile; amides, for example N,N-dimethylformamide, N,N-dimethylacetamide, N-methylformanilide, N-methylpyrrolidone, N-methylcaprolactam or hexamethylphosphoramide; sulphoxides, for example dimethyl sulphoxide, sulphones, for example tetramethylenesulphone, polyethers, for example 1,4-dioxane, ethylene glycol dimethyl ether, ethylene glycol diethyl ether, diethylene glycol dimethyl ether or diethylene glycol diethyl ether, or mixtures of such organic solvents.

The maximum water content of the solvent is preferably 1% by weight, more preferably 0.2% by weight and more preferably 0.05% by weight. Preference is given to attaining such a water content by incipient distillation or drying in a manner known per se. When alkali metal fluorides are used, particular preference is given to drying or incipiently distilling the solvent in the simultaneous presence of the alkali metal fluoride used.

The reaction temperature in the course of the halogen exchange may be, for example, 60° C. up to the boiling point of the solvent used at reaction pressure, but a maximum of 300° C., preferably 110° C. up to the boiling point of the solvent used at reaction pressure, and to a maximum of 200° C.

The reaction pressure may be, for example, 0.5 to 100 bar, and preferably 3 to 25 bar.

The reaction time may be, for example, 10 min to 72 hours, and preferably 2 to 12 hours.

The compounds of the formula (I) obtainable in accordance with the invention are suitable in particular in a process for preparing active ingredients, for example active ingredients for agrochemicals, such as in particular insecticides of the aroylurea type. Particularly preferred insecticides of the aroylurea type are those specified in EP-A 919 542 and EP-A 936 212.

It has been found that, surprisingly, the compounds of the formula (XI) which can be prepared particularly advantageously by the process according to the invention are particularly suitable as starting materials for the preparation of highly effective insecticides.

Therefore, the invention likewise encompasses the compounds of the formula (XI). In formula (XI)

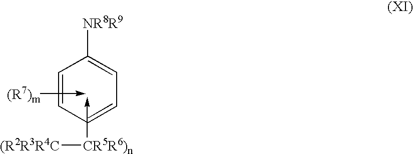

R², R³, R⁴, R⁵, R⁶, R⁷, R⁸ and R⁹, and m and n, each have the same definitions and areas of preference as already specified under formula (I), with the proviso that either

the R²R³R⁴C—CR⁵R⁶ radical, based on the carbon frameworks, is a secondary or tertiary radical or

is a primary radical which is selected from the group of 2-bromo-1,1,2,2-tetrafluoroethyl, 2-chloro-1,1,2,2-tetrafluoroethyl, 2-bromo-2chlorotrifluoroethyl and 2-bromo-1-chlorotrifluoroethyl,

and also excluding compounds in which R²R³R⁴C—CR⁵R⁶ as a whole is a perfluoroalkyl radical.

The scope of the claimed invention also encompasses compounds of the formula (XII)

(XIa)·(HY)ᵥ

in which

(XIa) represents compounds of the formula (XI) which have at least one primary, secondary or tertiary amino function and

v is a number in the range from 1 to the number of the primary, secondary or tertiary amino functions in the molecule (XIa) and

Y is an anion.

Y is preferably chlorine, bromine and hydrogen sulphate.

Preference is further given to those compounds of the formula (XIa) in which NR⁸R⁹ as a whole is a primary, secondary or tertiary amino radical and the compound of the formula (XIa) has no further primary, secondary or tertiary amino radicals.

A significant advantage of the process according to the invention is that the compounds of the formula (I) can be obtained in a simple manner in high yields from readily available reactants. Moreover, the compounds of the formula (XI) and (XII) constitute valuable starting products for the preparation of active ingredients, in particular for agrochemicals.

### EXAMPLES

### Example 1

Preparation of 1,2-dibromohexafluoropropane

2357 g of bromine (760 ml, 14.75 mol) were initially charged at room temperature and hexafluoropropene was introduced with constant stirring until decolorization (19 hours, 2400 g, 16.00 mol). The reaction mixture was purged with nitrogen. In this way, 4710 g of 1,2-dibromohexafluoropropane (95% of theory) were obtained.

### Example 2

Preparation of 2-bromoheptafluoropropane

An autoclave was initially charged with tetramethylenesulphone (2450 ml) and 352 g of potassium fluoride (6.05 mol) and the mixture was dried by distilling off 250 ml of solvent. Subsequently, 1250 g of 1,2-dibromohexafluoropropane from Example 1 were added, and the mixture was placed under 3 bar of nitrogen and heated to 125° C., resulting in a pressure of 13.5 bar. Heating was continued at the same temperature for a further two hours and then the temperature was increased to 175° C. within three hours. The mixture was cooled to 0° C., the autoclave decompressed and the product distilled out of the reaction mixture into a cold trap. In this way, 870 g of 2-bromoheptafluoropropane having a purity of 95.8% were obtained (83% of theory).

### Example 3

Preparation of 2-methyl-4-(1,1,1,2,3,3,3-heptafluoro-2-propyl)anilin

First 324.96 g (1.87 mol) of sodium dithionite and then 100 g (0.93 mol) of otoluidine (2-methylaniline) were added at room temperature to a mixture of 1200 ml of water, 250 ml of tert-butyl methyl ether, 156.79 g (1.87 mol) of sodiumhydrogen carbonate and 22.18 g of tetra-n-butylammonium hydrogensulphate. Subsequently, a solution of 489.06 g of 2-bromo-1,1,1,2,3,3,3-heptafluoropropane in 200 ml of tert-butyl methyl ether was added dropwise and, on completion of addition, the mixture was stirred at 30° C. overnight. If necessary, sodium carbonate was used to adjust to a pH of 5 and the organic phase was removed, dried and concentrated.

In this way, 250 g (90% of theory) of the product having a purity of 93% were obtained.

### Example 4

Preparation of N,2-dimethyl-4-(1,1,1,2,3,3,3-heptafluoro-2-propyl)aniline

In a similar manner to Example 3, the product was obtained in high yield and purity starting from N,2-dimethylaniline.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for preparing compounds of the formula (I) 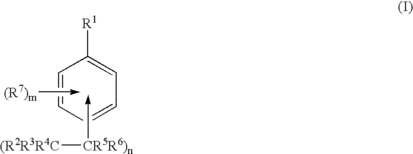
in which
R¹ is C₁-C₁₂-alkyl, NR⁸R⁹ or OR¹⁰, where R⁸, R⁹ and R¹⁰ are each independently hydrogen, C₁-C₁₂-alkyl, CO(C₁-C₁₂-alkyl), CO(C₅-C₁₄-aryl), CO(C₆-C₁₅-arylalkyl), COO(C₁-C₁₂-alkyl), COO(C₅-C₁₄-aryl), COO(C₆-C₁₅-arylalkyl), COO(C₂-C₁₂-alkenyl), CONH(C₁-C₁₂-alkyl), CONH(C₅-C₁₄-aryl), CONH(C₆-C₁₅-arylalkyl), CON(C₁-C₁₂-alkyl)₂, CON(C₅C₁₄-aryl)₂, CON(C₆-C₁₅-arylalkyl)₂ or C₆-C₁₅-arylalkyl, or NR⁸R⁹ as a whole is a cyclic radical having a total of 4 to 16 carbon atoms and
R², R³, R⁴, R⁵ and R⁶ are each independently hydrogen, fluorine, chlorine, bromine or C₁-C₁₂-polyfluoroalkyl, and/or at least two of the R², R³, R⁴, R⁵ and R⁶ radicals form one or more cyclic polyfluoroalkyl radicals each having a total of 4 to 20 carbon atoms, with the proviso in all cases that the sum of the fluorine atoms on the carbon atom which forms the bond to the aromatic ring and the fluorine atoms at the adjacent carbon atom or atoms is at least two and
n is one or two, and
R⁷ is C₁-C₁₂-alkyl, C₅-C₁₄-aryl, C₆-C₁₅-arylalkyl, hydroxyl, chlorine, bromine, fluorine, nitro, cyano, free or protected formyl, C₁-C₁₂-haloalkyl, or radicals of the formulae (IIa) to (IIf),
A—B—D—E (IIa)
A—E (IIb)
A—SO₂—E (IIc)
A—B—SO₂R¹¹ (IId)
A—SO₃W (IIe)
A—COW (IIf)
in which, each independently,
A is absent or is a C₁-C₈-alkylene radical and
B is absent or is oxygen, sulphur or NR¹²,
where R¹² is hydrogen, C₁-C₈-alkyl, C₆-C₁₅-arylalkyl or C₅-C₁₄-aryl, and
D is a carbonyl group and
E is R¹³, OR¹³, NHR¹¹ or N(R¹¹)₂
where R¹³ is C₁-C₈-alkyl, C₆-C₁₅-arylalkyl or C₅-C₁₄-aryl, and
R¹¹ is in each case independently C₁-C₈-alkyl, C₆-C₁₅-arylalkyl or C₆-C₁₄-aryl, or N(R¹¹)₂ together is a cyclic amino radical having 4 to 12 carbon atoms and
W is OH, NH₂ or OM where M is an alkali metal ion, half an equivalent of an alkali earth metal ion, an ammonium ion or an organic ammonium ion, or
two R⁷ radicals together, in each case, optionally form a cyclic radical having a total of 5 to 12 carbon atoms, and
m is an integer from 0 to 5-n,
comprising, reacting compounds of the formula (II) 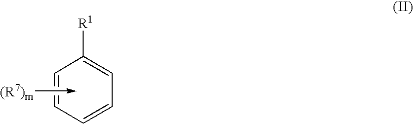
in which
R¹, R⁷ and m are each as defined above with compounds of the formula (III) 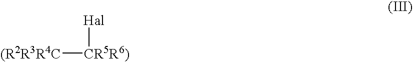
in which
R², R³, R⁴, R⁵ and R⁶are each as defined above and
Hal is bromine or chlorine, wherein the reaction is effected
in a multiphasic reaction medium which has one aqueous phase and at least one,
in the presence of phase transfer catalyst and
in the presence of a reducing agent and/or light having a wavelength of 400 nm or less.

2. Process according to claim 1, characterized in that it is carried out in the presence of base.

3. Process according to claim 2, characterized in that the base used is an alkali metal or alkaline earth metal hydroxide, acetate, phosphate, hydrogenphosphate, carbonate or hydrogencarbonate, ammonium salt, amine or aromatic nitrogen compound.

4. Process according to claim 1, characterized in that R¹ is NR⁷R⁸.

5. Process according to claim 1, characterized in that R², R³, R⁴ and R⁵ are each hydrogen, chlorine, fluorine or C₁-C₄-perfluoroalkyl, or R²R³R⁴C—CR⁵R⁶ as a whole is a cyclic polyfluoroalkyl radical having a total of 4 to 12 carbon atoms.

6. Process according to claim 1, characterized in that n is 1.

7. Process according to claim 1, characterized in that R⁷ is in each case independently C₁-C₄-alkyl, chlorine, fluorine, nitro, cyano or C₁-C₄-alkoxy.

8. Process according to claim 1, characterized in that m is 1 or 2.

9. Process according to claim 1, characterized in that the compounds of formula (I) are 2-methyl-4-(heptafluoro-2-propyl)aniline, N,2-dimethyl-4-(1,1,1,2,3,3,3-heptafluoro-2-propyl)aniline, 2-methyl-4-(1-bromo-1,1,2,3,3,3-hexafluoro-2-propyl)aniline, 2-methyl-4-(2-bromo-1,1,2,2-tetrafluoroethyl)aniline, 2-methyl-4-(2-chloro-1,1,2,2-tetrafluoroethyl)aniline, 2-methyl-4-(1-chloro-1,1,2,3,3,3-hexafluoro-2-propyl)aniline, 2-methyl-4-(2-bromo-2-chlorotrifluoroethyl)aniline, 2-methyl-4-(2-bromo-1-chlorotrifluoroethyl)aniline, 2-methyl-4-(3-bromo-2,3-dichloro-1,1,1,4,4,4-hexafluoro-2-butyl)aniline, 2-methyl-4-(2-chloro-3,3,4,4-tetrafluorocyclobutyl)aniline, 2-methyl-4-(2-chloro-3,3,4,4,5,5-hexafluorocyclopentyl)aniline, 2-methyl-4-(2-bromo-3,3,4,4-tetrafluorocyclobutyl)aniline, 2-methyl-4-(2-bromo-3,3,4,4,5,5-hexafluorocyclopentyl)aniline, 2-methyl-4-(1-bromo-1,1,2,3,3,3-hexafluoro-2-propyl)acetanilide, 2-methyl-4-(2-bromo-1,1,1,2,3,4,4,4-octafluoro-3-butyl)aniline or 2-methyl-4-(2-bromo-2,3,3,4,4,5,5-octafluorocyclo-1-pentyl)aniline.

10. Process according to claim 1, characterized in that the compounds of the formula (III) are heptafluoro-2-bromopropane, heptafluoro-2-chloropropane, 1,2-dibromotetrafluoroethane, 1,2-dibromo-1-chlorotrifluoroethane, 2,3-dibromooctafluorobutane, 2,3-dibromo-2,3-dichlorohexafluorobutane, 2,3-dibromo-2,3-dichlorohexafluorobutane, 2,3-dibromo-1,1,1,3,4,4,4-heptafluorobutane, 2,3-dibromo-2-chloro-1,1,1,4,4,4-hexafluorobutane, 1,2-dibromohexafluoropropane and 1,2-dichlorohexafluoropropane.

11. Process according to claim 1, characterized in that the organic solvent used for muliphasic reaction media is an aliphatic or aromatic, optionally halogenated hydrocarbon, ether, ketone, or ester.

12. Process according to claim 1 characterized in that the phase transfer catalysts used are crown ethers such as 18-crown-6, 12-crown-4, dibenzo-18-crown-6 or dibenzo-12-crown-4, cryptands such as cryptand[2.2.2] or podands such as polyglycol ethers or those of the formula (IV),
(cation⁺)(anion⁻) (IV)
in which
(cation⁺) is a substituted quaternary ammonium or phosphonium cation and
(anion⁻) is the anion of an organic or inorganic acid.

13. Process according to claim 12, characterized in that the phase transfer catalysts used are those of the formula (IV) in which (cation⁺) is a cation of the formula (V)
[pnic(C₁-C₁₂-alkyl)_{q}(C₆-C₁₅-arylalkyl)ᵣ(C₅-C₁₄-aryl)ₛ({(C₂-C₆-alkyl)-O]ᵥ—(C₁-C₆-alkyl)}ₜ)]⁺ (V)
in which
pnic is nitrogen or phosphorus and
in which in each case (q+r+s+t)=4.

14. Process according to claim 13, characterized in that the reaction temperature is −10° C. up to the boiling point of the reaction medium under reaction pressure, and up to a maximum of 200° C., under reaction pressure of 0.5 to 100 bar.

15. Process according to claim 1, characterized in that the reducing agent used is a sulphur compound in the average formal oxidation states +III, +IV and +V, optionally in a mixture with a metal which has a standard reduction potential of 0 V or less.

16. Process according to claim 1 characterized in that alkali metal dithionites are used.

17. Process according to claim 1, characterized in that, in a subsequent step, compounds of the formula (I) in which the R²R³R⁴C—CR⁵R⁶ radical bears at least one chlorine or bromine atom are reacted with ionic fluoride to give compounds of the formula (I) in which at least one chlorine or bromine atom has been replaced by a fluorine atom.

18. Process according to claim 17, characterized in that the ionic fluorides used are quaternary ammonium fluorides or phosphonium fluorides, or alkali metal fluorides or mixtures of the compounds mentioned or mixtures of phase transfer catalysts and/or halex catalysts with alkali metal fluorides.

19. Process according to claim 17, characterized in that the halogen exchange is carried out in the presence of organic solvent selected from the group consisting of ketones, nitriles, amides, sulphoxides, sulphones, polyethers and mixtures thereof.

20. Compounds of the formula (XI) 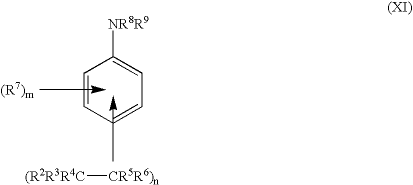
in which
R², R³, R⁴, R⁵, R⁶, R⁷, R⁸ and R⁹ and also m and n, are defined in claim 1, with the proviso that either
the R²R³R⁴C—CR⁵R⁶ radical, based on the carbon framework, is a secondary or tertiary radical or
is a primary radical which is selected from the group consisting of 2-bromo-1,1,2,2-tetrafluoroethyl, 2-chloro-1,1,2,2-tetrafluoroethyl, 2-bromo-2-chlorotrifluoroethyl and 2-bromo-1-chlorotrifluoroethyl,
excluding compounds in which R²R³R⁴C—CR⁵R⁶ as a whole is a perfluoroalkyl radical.

21. Compounds of formula (I) selected from the group consisting of 2-methyl-4-(1-bromo-1,1,2,3,3,3-hexafluoro-2-propyl)aniline, 2-methyl-4-(2-bromo-1,1,2,2-tetrafluoroethyl)aniline, 2-methyl-4-(2chloro-1,1,2,2-tetrafluoroethyl)aniline, 2-methyl-4-(1-chloro-1,1,2,3,3,3-hexafluoro-2-propyl)aniline, 2-methyl-4-(2-bromo-2-chlorotrifluoroethyl)aniline, 2-methyl-4-(2-bromo-1-chlorotrifluoroethyl)aniline, 2-methyl-4-(3-bromo-2,3-dichloro-1,1,1,4,4,4-hexafluoro-2-butyl)aniline, 2-methyl-4-(2-chloro-3,3,4,4-tetrafluorocyclobutyl)aniline, 2-methyl-4-(2-chloro-3,3,4,4,5,5-hexafluorocyclopentyl)aniline, 2-methyl-4-(2-bromo-3,3,4,4tetrafluorocyclobutyl)aniline, 2-methyl-4-(2-bromo-3,3,4,4,5,5-hexafluorocyclopentyl)aniline, 2-methyl-4-(1-bromo-1,1,2,3,3,3-hexafluoro-2-propyl)acetanilide, 2-methyl-4-(2-bromo-1,1,1,2,3,4,4,4-octafluoro-3-butyl)aniline and 2-methyl-4-(2-bromo-2,3,3,4,4,5,5-octafluorocyclo-1-pentyl)aniline.

22. Compounds of the formula (XII)
(XIa)·(HY)ᵥ
in which
(XIa) represents compounds of the formula (XI) according to claim which have at least one primary, secondary or tertiary amino function and
v is a number in the range from 1 to the number of the primary, secondary or tertiary amino functions in the molecule (XIa) and
Y is an anion.

23. A process for preparing active ingredients comprising incorporating compounds of formula (I) which have been prepared according to claim 1.

24. The process according to claim 23, characterized in that the active ingredients are insecticides.
